(19) United States
(12) Patent Application Publication
(10) Pub. No.: US 2026/0020873 A1
(43) Pub. Date: Jan. 22, 2026

(54) Tissue Disrupter

(71) Applicant: Stryker Corporation, Portage, MI (US)

(72) Inventors: Joseph L. Mark, Indianapolis, IN (US); Brian C. Dougherty, Terre Haute, IN (US)

(73) Assignee: Stryker Corporation, Portage, MI (US)

(21) Appl. No.: 19/274,484

(22) Filed: Jul. 18, 2025

Related U.S. Application Data
(60) Provisional application No. 63/672,993, filed on Jul. 18, 2024.

(51) Int. Cl. A61B 17/32; A61B 17/56

(52) U.S. Cl. CPC A61B 17/320016; A61B 17/56; A61B 2017/320008; A61B 2017/564

(57) ABSTRACT

A tissue disrupter assembly includes an outer cannula and an inner member arranged at least partially within the outer cannula. The outer cannula is selectively movable along the inner member to expose a tissue disrupter fixed to and extending from a distal end of the inner member. The tissue disrupter when exposed is configured to enter an expanded configuration for dislodging tissue at a surgical site.

### RELATED APPLICATION(S)

This application claims priority to and all the benefits of U.S. Provisional Patent Appl. No. 63/672,993 filed Jul. 18, 2024, the entire disclosure of which is hereby incorporated by reference herein.

### TECHNICAL FIELD

The present disclosure relates generally to a tissue disrupter assembly for use with a surgical access system.

### BACKGROUND

Spinal conditions, such as degenerative discs diseases, stenosis, herniated discs, etc., can cause severe pain for patients. Treatment for such conditions often entails the removal of disc material from the patient's spine. In the example of a herniated disc, the inner gel-like core (nucleus pulposus) of an intervertebral disc pushes through a tear in the outer layer (annulus fibrosus). This can cause pain, numbness, or weakness if the herniated material compresses nearby spinal nerves, and removal of some or all of the nucleus material may be performed to alleviate these symptoms. As a further example, a spinal fusion is a surgical procedure that joins two or more vertebrae permanently, often through the use of an implant placed between the vertebrae. This aims to eliminate motion between the vertebrae, providing stability and reducing pain. Prior to placing the implant which can be used to fuse the vertebrae, some or all of the disc therebetween is typically desired to be removed to prepare the space where the implant will be placed.

### SUMMARY

One general aspect includes a tissue disrupter assembly for treating nucleus tissue of a spinal disc while reducing disruption to annulus tissue of the spinal disc. The tissue disrupter assembly includes an outer cannula defined by a first open distal end, an opposite first proximal end, and a hollow body portion extending therebetween. The assembly also includes an inner member arranged at least partially within the outer cannula and having a second distal end and an opposite second proximal end. The assembly also includes a tissue disrupter configured to dislodge tissue at a surgical site, the tissue disrupter fixed to and extending from the second distal end of the inner member. The outer cannula is selectively and axially movable relative to the inner member to adjust the tissue disrupter between a first state in which the tissue disrupter is in a collapsed configuration and arranged entirely within the outer cannula, and a second state in which the tissue disrupter is in an expanded configuration and positioned distal to the first open distal end. The tissue disrupter forms a hoop in the expanded configuration, the hoop having at least one cutting edge to dislodge the nucleus tissue and a blunt distal curvature portion to reduce disruption of the annulus while dislodging the nucleus tissue.

A further general aspect includes a tissue disrupter assembly having an outer cannula defined by a first open distal end, an opposite first proximal end, and a hollow body portion extending therebetween. The assembly also includes an inner member arranged at least partially within the outer cannula and having a second distal end and an opposite second proximal end. The assembly also includes a tissue disrupter configured to dislodge tissue at a surgical site, the tissue disrupter fixed to and extending from the second distal end of the inner member. The outer cannula is selectively and axially movable relative to the inner member to adjust the tissue disrupter between a first state in which the tissue disrupter is in a collapsed configuration and arranged entirely within the outer cannula, and a second state in which the tissue disrupter is in an expanded configuration and positioned distal to the first open distal end. The tissue disrupter includes a plurality of malleable protrusions each extending distally from the second distal end of the inner member and terminating in a blunt distal end when the tissue disrupter is in the expanded configuration.

A further general aspect includes a method for treating nucleus tissue of a spinal disc while reducing disruption to annulus tissue of the spinal disc. The method includes providing an access portal to the nucleus tissue of the spinal disc. The method also includes obtaining a tissue disrupter assembly having an outer cannula defined by a first open distal end, an opposite first proximal end, and a hollow body portion extending therebetween; an inner member arranged at least partially within the outer cannula and having a second distal end and an opposite second proximal end; and a tissue disrupter fixed to and extending from the second distal end of the inner member, with the tissue disrupter being in a first state in which the tissue disrupter is in a collapsed configuration and arranged entirely within the outer cannula. The method also includes inserting the obtained tissue disrupter assembly in the first state through the access portal such that the first open distal end of the outer cannula passes through the annulus tissue and is disposed in the nucleus tissue. The method also includes adjusting the tissue disrupter to a second state in which the tissue disrupter is in an expanded configuration and positioned distal to the first open distal end by moving the outer cannula relative to the inner member in a proximal direction, where the tissue disrupter forms a hoop in the expanded configuration that has at least one cutting edge to dislodge the nucleus tissue and a blunt distal curvature portion to reduce disruption of the annulus while dislodging the nucleus tissue. The method also includes dislodging the nucleus tissue from vertebrae adjacent the spinal disc by moving the tissue disruption assembly in the second state within the spinal disc. The method also includes after dislodging the nucleus tissue, adjusting the tissue disrupter back to the first state by moving the outer cannula relative to the inner member in a distal direction. The method also includes withdrawing the tissue disrupter back in the first state from the access portal. The method also includes removing the dislodged nucleus tissue from the spinal disc using a tissue removal device inserted through the access portal.

### BRIEF DESCRIPTION OF THE DRAWINGS

Exemplary features of the present disclosure will now be described in greater detail with reference to the attached figures, in which:

FIG. 1 illustrates a side view of an endoscope inserted through an annulus of a spine.

FIG. 2 illustrates another side view of the spine with a portion of the endoscope in phantom.

FIG. 3 illustrates a perspective view of a tissue disrupter assembly in a collapsed state.

FIG. 4 illustrates a perspective view of a portion of the tissue disrupter assembly of FIG. 3.

FIG. 5 illustrates a perspective view of the tissue disrupter assembly in an expanded state.

FIG. 6 illustrates a side view of the tissue disrupter assembly inserted into the endoscope and entering the annulus in a collapsed state.

FIG. 7 illustrates a side view of the distal end of the tissue disrupter assembly of FIG. 6.

FIG. 8 illustrates a side view of the tissue disrupter assembly inserted into the endoscope and dislodging the disc in an expanded state.

FIG. 9 illustrates a side view of the distal end of the tissue disrupter assembly of FIG. 8.

FIG. 10 illustrates a side view of the distal end of the tissue disrupter assembly of FIGS. 8 and 9.

FIG. 11 illustrates a side view of the tissue disrupter assembly in a collapsed state for being withdrawn from the spine and the endoscope.

FIG. 12 illustrates a side view of the distal end of the tissue disrupter assembly of FIG. 11.

FIG. 13 illustrates a side view of a tissue removal device inserted into the endoscope to remove dislodged tissue from the spine.

FIG. 14 illustrates a side view of the distal end of the tissue removal device of FIG. 13.

FIG. 15 illustrates a side view of a portion of the tissue removal device of FIG. 13.

FIG. 16 illustrates a perspective view of an example tissue disrupter that may be incorporated into the tissue disrupter assembly.

FIG. 17 illustrates a perspective view of another example tissue disrupter that may be incorporated into the tissue disrupter assembly.

FIG. 18 illustrates a perspective view of a further example tissue disrupter that may be incorporated into the tissue disrupter assembly.

FIG. 19 illustrates a perspective view of an additional example tissue that may be incorporated into the tissue disrupter assembly.

FIG. 20 illustrates a perspective view of another example tissue disrupter that may be incorporated into the tissue disrupter assembly.

FIG. 21 illustrates a perspective view of a further example tissue disrupter that may be incorporated into the tissue disrupter assembly.

FIG. 22 illustrates a side view of the tissue disrupter assembly having a ribbon-type tissue disrupter in a collapsed state.

FIG. 23 illustrates a side view of the tissue disrupter assembly of FIG. 22 in an expanded state.

### DETAILED DESCRIPTION

Referring now to the discussion that follows and also to the drawings, illustrative approaches to the disclosed assemblies and methods are provided in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive or otherwise limit or restrict the claims to the precise forms and configurations shown in the drawings and disclosed in the following detailed description.

Described herein is a surgical access assembly, various components for use in the same, and a method of using the surgical access assembly. The components disclosed herein provide surgeons with an enhanced ability to minimize trauma to the patient, while providing efficient improved minimally invasive surgical techniques.

Specifically, a tissue disrupter assembly is disclosed herein that is configured to aid in the removal of intervertebral disc related tissues, specifically including the nucleus pulposus situated between vertebrae in the spinal column. This gel-like substance is primarily composed of water, collagen, and proteoglycans. The nucleus pulposus functions as a shock absorber and provides for flexibility and cushion between the vertebrae to enable movements such as bending and twisting. The nucleus pulposus also helps to transport nutrients and waste products between the vertebral bodies and the intervertebral disc. The nucleus pulposus may degrade over time and be subject to injuries. This may lead to herniated discs or degenerative discs. A decrease in the height or volume of the nucleus pulposus can lead to chronic pain and mobility issues for patients.

Treatment of a herniated disc often involves the removal of some or all of the nucleus. In the case of what is termed a spinal fusion where adjacent vertebrae are to be fused, as much as possible all of the nucleus material is often desired to be removed, while in some procedures maintaining the integrity of the annulus. The spinal fusion procedure may permanently join adjacent vertebrae to eliminate motion therebetween, which may help reduce pain and improve stability. In some procedures, an implant or “jack” may be inserted between the vertebrae, such as in place of the removed nucleus material, to maintain a vertical height between the vertebrae and hold the vertebrae in place during the fusion process.

The removal of disc material during such procedures may be accomplished by a portal approach that may or may not include the use of a scope. The scope may be a working channel spinal scope, such as commercially available from companies such as Storz or Joimax. With the portal approach, a cannula may be inserted into the disc area, such as through the working channel spinal scope, from the ipsilateral or contralateral side of the spine. The use of the portal approach with a cannula creates a minimally invasive access to the site to allow for the procedure to be performed with reduced possibility of damage to the nerve roots exiting from the spinal column. Instruments may then be inserted through the portal/cannula to remove the intended portions of the disc such as the nucleus. The nucleus disc tissue is often tenaciously attached to the end plates of the adjacent vertebrae. This can cause difficulties in removing the nucleus from the upper and lower vertebrae substrate and the portions of the disc adjacent thereto.

In some cases, it is also desirable to maintain the annulus fibrosis while removing the nucleus disc tissue. The annulus fibrosis is a tough, outer layer of an intervertebral disc. This layer surrounds and protects the inner core of the nucleus pulposus. Removal of the nucleus of the disc from the vertebrae without compromise or disruption, or further compromise or disruption, of the annulus fibrosis can help promote an effective spinal fusion.

Various examples of a tissue disrupter assembly are disclosed herein. The tissue disrupter assembly may be a portal approach device for use, such as with a working channel of a scope, to dislodge the disc material from the adjacent vertebrae without necessitating repeated in and out motions from the surgical site. The tissue disrupter assembly may additionally or alternatively be configured to effectively scrape the nucleolus tissue from one or both of the end plates of the vertebrae. In some implementations, the tissue disrupter may be configured to scrape tissue from both end plates simultaneously.

For example, the tissue disrupter may include a flexible hoop configured to dislodge the nucleus disc tissues. The distal end of the formed hoop of the tissue disrupter may be blunt (not sharp) so as to not perforate or damage the annulus when in use, whereas the edges of the hoop may be sharpened. The tissue disrupter may be configured to manipulate and dislodge portions of the disc. The dislodged disc material may then be removed from the disc space by a tissue removal device, such as the MYRIAD device by Nico or other known tissue removal device, that generally allows for removal of the desired tissues via a single insertion of the device into the surgical site. This method minimizes the in and out motion relative to the surgical site, creating a more efficient and safer procedure.

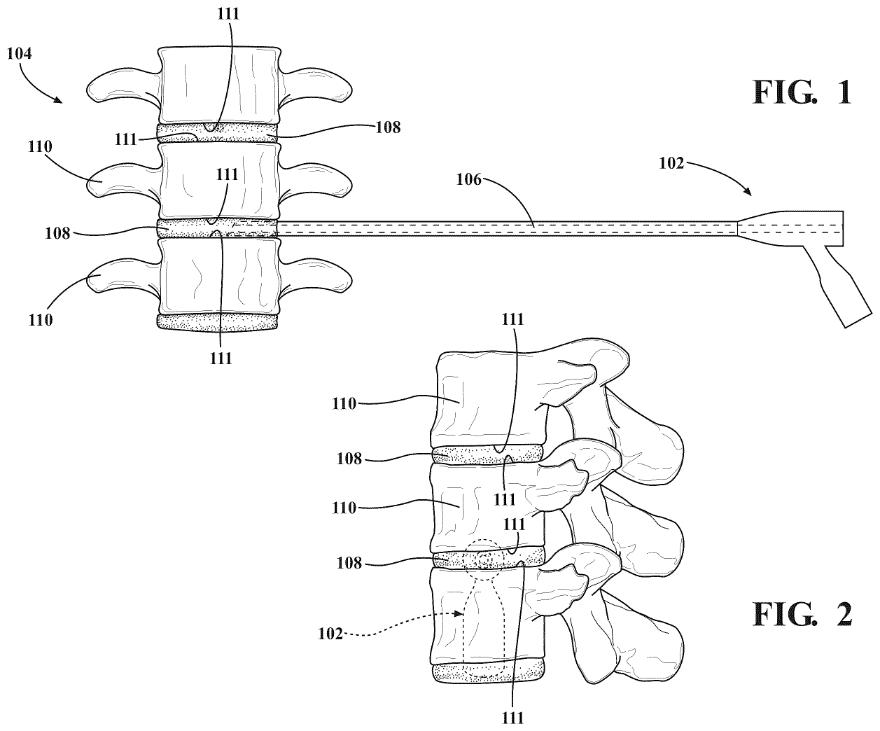

FIG. 1 illustrates a side view of an endoscope 102 inserted through an annulus of a spine 104. FIG. 2 illustrates another side view of the spine with a portion of the inserted endoscope 102 in phantom. The endoscope 102 may define a working channel 106 configured to receive surgical tools, such a probes, tissue disrupters, cannulas, resection devices, tissue removal devices, light sources, biopsy devices, to name a few. While the endoscope 102 and applications discussed herein are directed to spinal related procedures, other areas or tissues may benefit from the use of the technology discussed herein.

The endoscope 102 may be inserted into and/or through the annulus fibrosus of an intervertebral disc 108 (also referred to herein as disc tissue 108) of the spine 104. The annulus is a tough outer layer of the intervertebral disc 108 that encases the nucleus pulposus. The disc 108 may be arranged between two vertebrae 110, or more particularly between endplates 111 of the vertebrae 110. Once inserted, the endoscope 102 may allow for surgical tools to reach the disc 108 via the working channel 106.

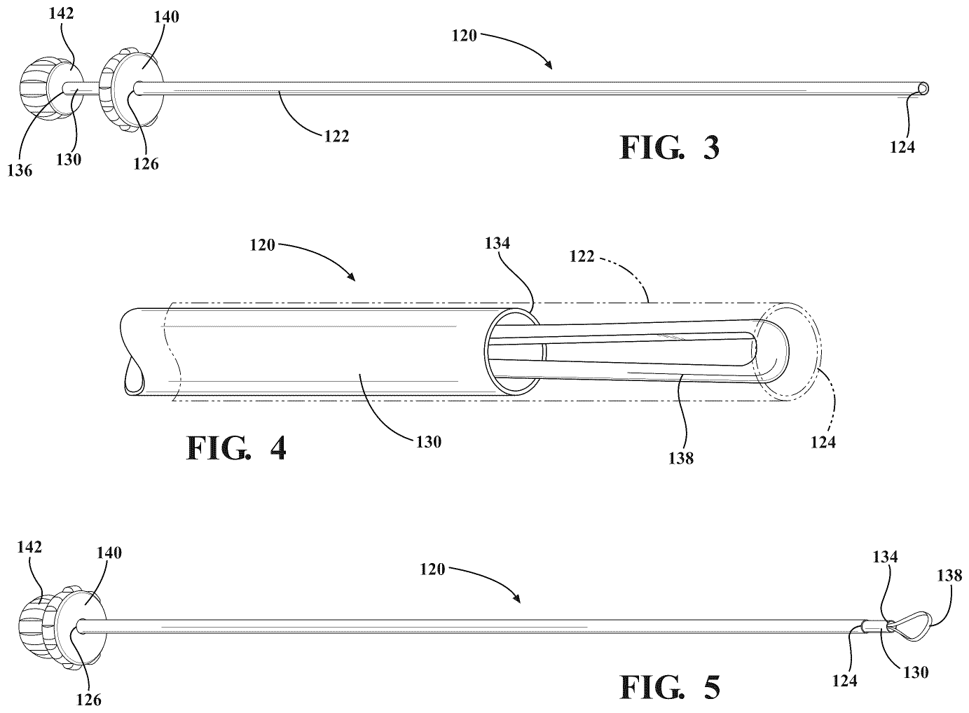

FIGS. 3-5 illustrate a tissue disrupter assembly 120 that may be inserted through the working channel 106 of the endoscope 102 to dislodge and/or scrape the targeted disc tissue. Specifically, FIG. 3 illustrates the tissue disrupter assembly 120 in a collapsed state, FIG. 4 illustrates a portion of the tissue disrupter assembly 120 of FIG. 3, and FIG. 5 illustrates a the tissue disrupter assembly 120 in an expanded state.

As shown in the illustrated examples, the tissue disrupter assembly 120 may include an outer cannula 122 defined by a first open distal end 124, a first proximal end 126 opposite the first open distal end 124, and a hollow body portion extending therebetween. An inner member 130, such as an inner cannula, may be arranged at least partially within the outer cannula 122 and have a second distal end 134, a second proximal end 136 opposite the second distal end 134, and an inner and/or at least partially hollow body portion extending therebetween. The outer cannula 122 may be selectively and axially movable along the inner cannula 130.

A tissue disrupter 138 may extend from the second distal end 134 of the inner cannula 130. The tissue disrupter 138 may be configured to dislodge and/or scrape tissue at a surgical site. For instance, the tissue disrupter 138 may be configured to dislodge and/or scrape material of the disc 108 between the vertebrae 110. The tissue disrupter 138 is discussed in more detail herein.

Still referring to FIGS. 3-5, a first hub 140 may be arranged at the first proximal end 126 of the outer cannula 122 and a second hub 144 may be arranged at the second proximal end 136 of the inner cannula 130. In the collapsed state, the first hub 140 may be spaced from the second hub 142, as shown in the illustrated example of FIG. 3. In this state, the first distal end 124 of the outer cannula 122 may extend past the second distal end 134 of the inner cannula 130 to cover the tissue disrupter 138, which in this state may be entirely arranged within the outer cannula 122 and held by the outer cannula 122 in a collapsed configuration. The outer cannula 122 may be configured to selectively move along the inner cannula 130 via actuation at one of the first hub 140 and the second hub 144 towards the other. The inner cannula 130 may be of greater length than the outer cannula 122 such that when the first hub 140 and the second hub 142 are moved towards one another, the first distal end 124 of the outer cannula 122 exposes at least a portion of the inner cannula 130 and the tissue disrupter 138 at the second distal end 134 of the inner cannula 130. In other words, the first hub 140 may be manually moved back towards the second hub 142 to expose the tissue disrupter 138, such as shown in the illustrated example of FIG. 5.

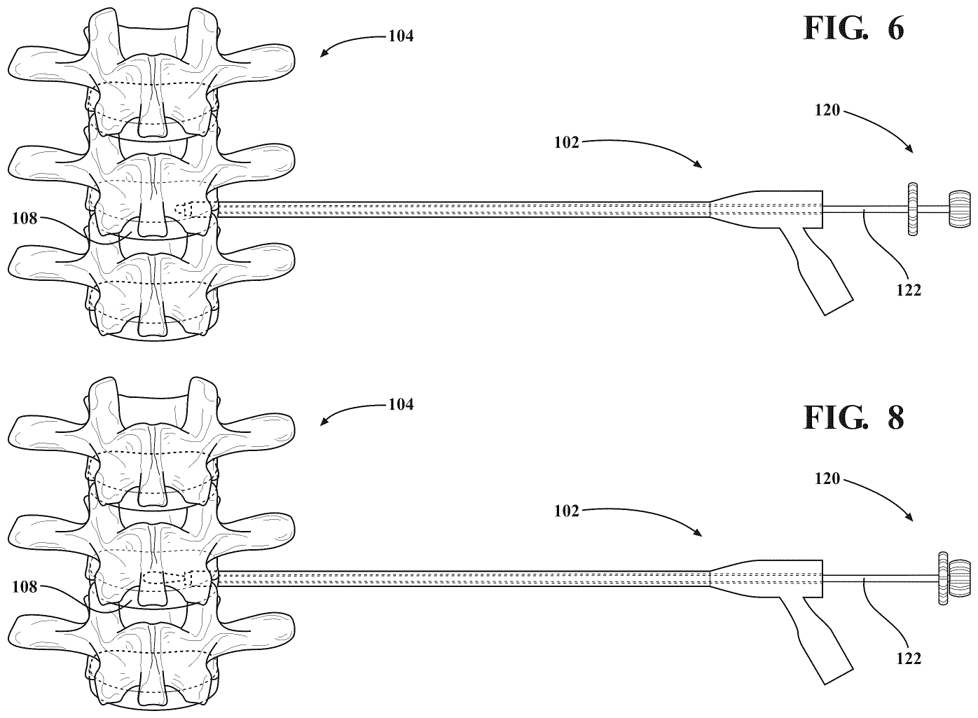

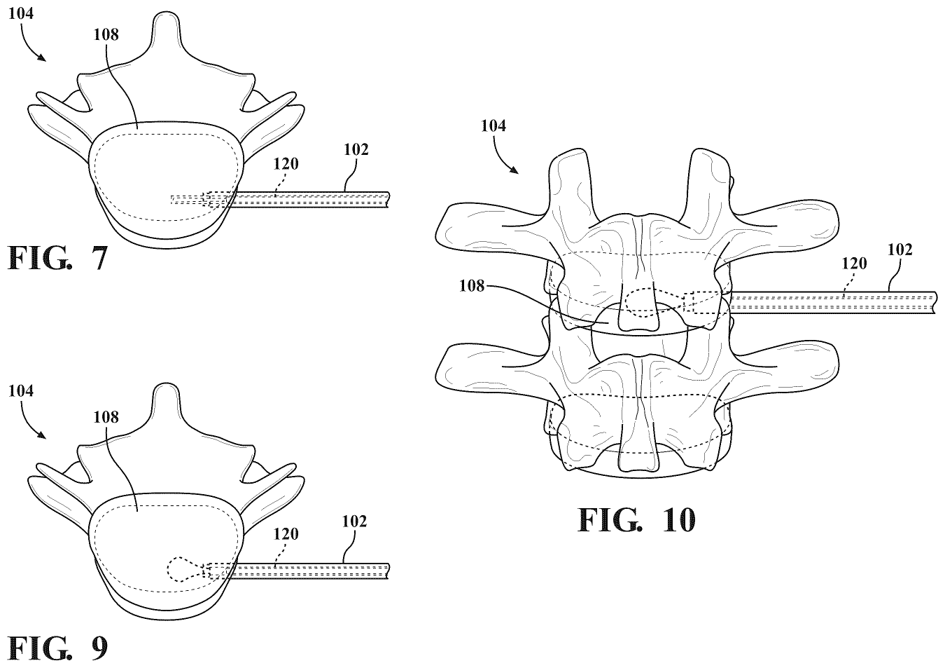

FIGS. 6-14 illustrate a process in which the tissue disrupter assembly 120 may be used to dislodge and/or scrape tissue of a disc 108 as described herein. FIG. 6 illustrates a side view of the tissue disrupter assembly 120 inserted through the endoscope 102 and the annulus of the disc 108 and in the collapsed state. FIG. 7 illustrates a top view of the distal end of the tissue disrupter assembly 120 in the collapsed state and positioned as in FIG. 6. FIG. 8 illustrates a side view of the tissue disrupter assembly 120 inserted through the endoscope 102 and the annulus of the disc 108 and in the expanded state to dislodge material of the disc 108. FIG. 9 illustrates a top view of the distal end of the tissue disrupter assembly 120 in the expanded state and positioned as in FIG. 9. FIG. 10 illustrates a side view of the distal end of the tissue disrupter assembly 120 in the expanded state and positioned as in FIGS. 8 and 9.

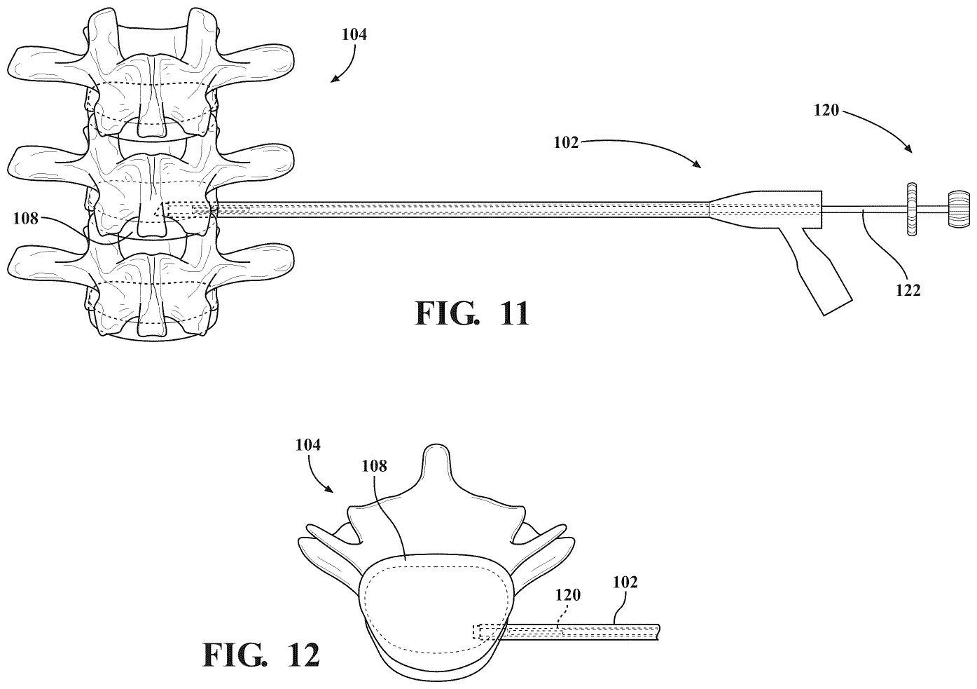

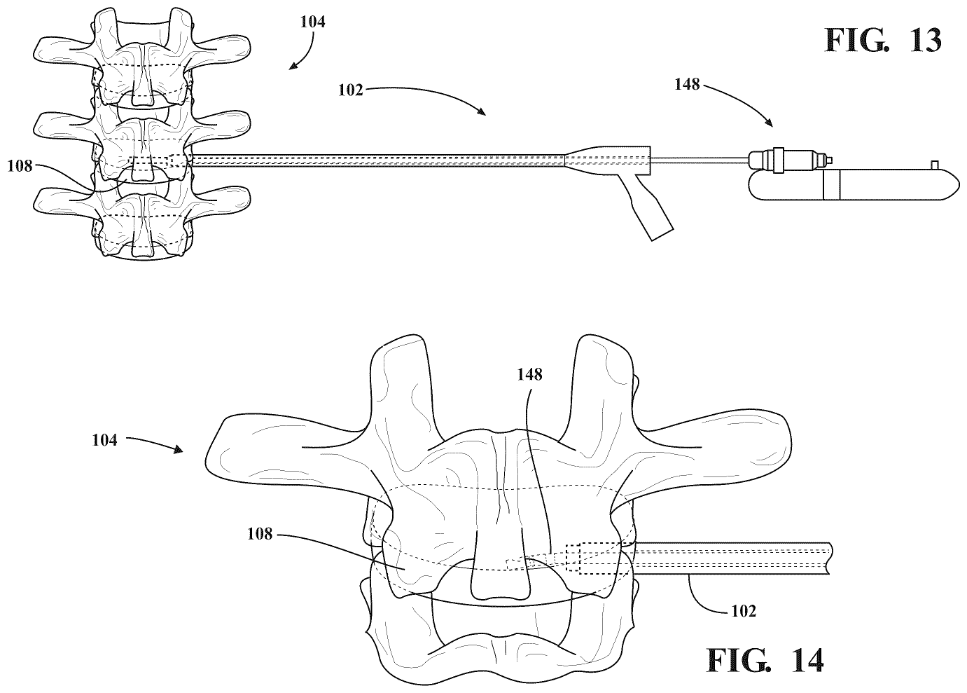

FIG. 11 illustrates a side view of the tissue disrupter assembly 120 inserted in the endoscope 102 and after being used to dislodge the tissue material of the disc 108, the tissue disrupter assembly 120 being again in the collapsed state for being removed from the patient and the endoscope 102. FIG. 12 illustrates a top view of the distal end of the tissue disrupter assembly 120 in the collapsed state and positioned as in FIG. 11. FIG. 13 illustrates a side view of a tissue removal device 148 inserted through the endoscope 102 and into the disc 108 to resect the dislodged tissue material from the disc. FIG. 14 illustrates a side view of the distal end of the tissue removal device 148 when positioned as in FIG. 13.

As illustrated in these Figures, during a procedure involving the removal of disc 108 material from a patient's spine, the endoscope 102 may be inserted into the disc 108. The tissue disrupter assembly 120 may then be inserted into the working channel 106. Prior to insertion into the working channel 106, the tissue disrupter assembly 120 may be configured in the collapsed state where the tissue disrupter 138 is in a collapsed configuration and housed within the outer cannula 122. Once the tissue disrupter assembly 120 is inserted through the working channel 106 and into the disc 108 as shown in FIGS. 6 and 7, the clinician may grip the first hub 140 and retract the first hub 140 back towards the second hub 142, thus moving the outer cannula 122 proximally along the inner cannula 130 to expose the tissue disrupter 138 at the distal end of the tissue disrupter assembly 120.

Upon retraction of the outer cannula 122, the tissue disrupter 138 may expand radially outwardly to form a circular or semi-circular shape, such as illustrated by way of example in FIGS. 8-10. To this end, the tissue disrupter 138 may be formed of a flexible and/or malleable material capable of retaining a shape, and/or collapsing under the pressure of the outer cannula 122. FIGS. 9 and 10 illustrate an example of the tissue disrupter 138 being in the fully expanded state. The tissue disrupter 138 may also be capable of bending around bone such as vertebrae. For instance, the flexibility of the tissue disrupter 138 allows for the tissue disrupter 138 to abut and flex to conform to the endplates 111 of the vertebrae 110. This movement allows for cleaner and further disruption and dislodging of the nucleus disc tissue 108. The disc tissue 108 may be dislodged from the vertebrae 110 using the tissue disrupter 138 in large sections as well as in smaller pieces. The vertebrae 110, or more particularly the endplates 111 of the vertebrae 110, may act as a guide during the dislodging. The tissue disrupter 138 may be rotated and moved within the disc space to continue to cut parts of the nucleus away. The tissue disrupter 138 may remain within the spine, or more particularly within the nucleus tissue of the disc 108, until the desired amount of tissue has been dislodged and/or removed. Such feature reduces the number of insertions of instruments into and removal of instruments from the surgical site, helping to decrease risk of nerve damage typically caused by in and out motions near the spinal nerves. Notably, the tissue disrupter 138 may also be used without engaging the annulus.

Once the desired amount of particles or masses are dislodged and/or cut away from the vertebrae 110, the tissue disrupter assembly 120 may be removed from the surgical site such as via the endoscope 102, and a device such as a tissue removal device 148 may be inserted into the surgical site to evacuate the dislodged masses. Prior to removing the tissue disrupter assembly 120 from the endoscope 102, the tissue disrupter 138 may be retracted into the collapsed state within the outer cannula 122 again, such as illustrated in FIGS. 11 and 12. Once the tissue disrupter 138 is “stored” within the outer cannula 122, the tissue disrupter assembly 120 may then be removed from the working channel 106 of the endoscope 102.

As illustrated in FIGS. 13 and 14, the tissue removal device 148 may then be inserted through the working channel 106 of the endoscope 102 and into the surgical site. The tissue removal device 148 may then be configured to remove in situ the masses or pieces dislodged by the tissue disrupter 138.

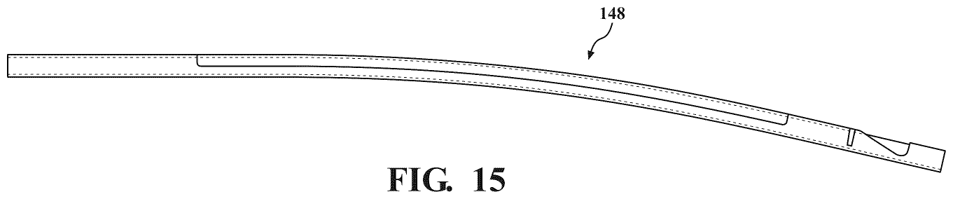

FIG. 15 illustrates a portion of the tissue removal device 148 of FIGS. 13 and 14 according to one example. The tissue removal device 148 may be curved, flexible or rigid, and may include an opening configured to receive the disc masses therein. The tissue removal device 148 may be easily moved between and within the vertebral endplates 111 to remove the dislodged masses of tissue without the need to place the tissue removal device 148 in and out of the working channel 106 which accesses the surgical site. As one example, the tissue removal device 148 may configured similarly to that shown in U.S. Pat. No. 8,888,803, the entire contents of which is hereby incorporated by reference herein.

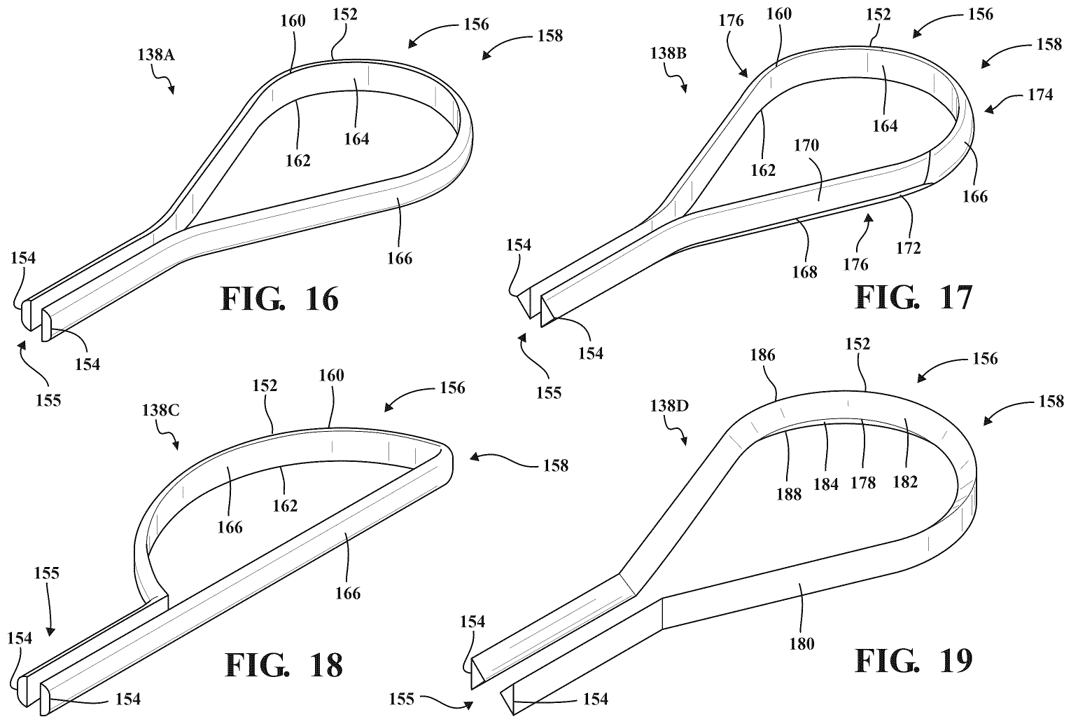

FIGS. 16-21 illustrate various examples of tissue disrupters 138 that may be used in the tissue disrupter assembly 120. As shown by the various examples, some edges of the tissue disrupter 138 may form relatively sharp edges, which may facilitate cutting of the disc 108 tissue. In some examples, the tissue disrupter 138 may form a hoop including one or more internal and/or external sharp cutting edges, but with a most distal curvature portion of the hoop lacking a sharp edge. Omitting a sharp edge from the distal curvature of the tissue disrupter 138 may help prevent cutting or disruption of the annulus during dislodging of the nucleus tissue. That is, the tissue disrupter 138 may be moved, rotated, etc. within the nucleus, but because the distal most point of the hoop is not sharp, even if the tissue disrupter 138 abuts, scrapes, or otherwise contacts the annulus during the procedure, the annulus may stay intact and unharmed. The cross-sectional shape or edges of the tissue disrupter 138 may also vary, as described in more detail below.

FIG. 16 illustrates an example tissue disrupter 138A including an elongated body 152 extending between opposed terminal ends 154. The elongated body 152 may be molded into a shape in which the terminal ends 154 are disposed proximate one another to form a proximal end portion 155 of the tissue disrupter 138A, and a portion of the elongated body 152 extending between the terminal ends 154 may form a hoop 156 having a circular shape, which in turn may form a distal end portion 158 of the tissue disrupter 138A. The hoop 156 of the tissue disrupter 138A may be configured to elongate in the proximal-distal direction when the tissue disrupter 138A is placed in the collapsed configuration, such as responsive to the outer cannula 122 being slid over the tissue disrupter 138A as described above, so as enable the tissue disrupter 138A to be arranged within the outer cannula 122, such as for easy insertion of the tissue disrupter assembly 120 into the surgical site.

The tissue disrupter 138A may define an upper inner edge 160 and a lower inner edge 162, each which may extend along an inner perimeter of the hoop 156. More specifically, the tissue disrupter 138A may include an inward facing surface 164 defining the inner perimeter of the hoop 156 and an outward facing surface 166 defining an outer perimeter of the hoop 156. The outward facing surface 166 may intersect the top and bottom of the inward facing surface 164 along the inner perimeter of the hoop 156 to define the upper and lower inner edges 160, 162. In some implementations, the upper and lower inner edges 160, 162 may be sharpened cutting edges, and/or may be configured with a positive rake ankle (e.g., the inward facing surface 164 may have a concave profile in the direction generally parallel to a central axis of the hoop 156). As shown in the illustrated example, the outward facing surface 166 may have a curved profile from the upper inner edge 160 to the lower inner edge 162 so as to provide the tissue disrupter 138, including its most distal curvature portion, with a relatively blunt outer perimeter. For instance, the transverse cross-sectional shape of the elongated body 152 may be a semi-circle with the circular portion forming outward facing surface 166 of the hoop 156.

FIG. 17 illustrates another example tissue disrupter 138B that similarly includes a hoop 156 forming a circular shape at the distal end portion 158, with the hoop 156 likewise including upper and lower inner edges 160, 162. As shown in the illustrated example, the tissue disrupter 138B may also include an outer cutting edge 168 disposed along at least a portion of the outer perimeter of the hoop 156 of the tissue disruption 138B and extending radially outward. For instance, the hoop 156 of the tissue disrupter 138B may include an upper outward facing surface 170 and a lower outward facing surface 172 each extending along the outer perimeter of the hoop 156 and extending outwardly from the upper inner edge 160 and the lower inner edge 162 to define the outer cutting edge 168. The portion of the elongated body 152 of the tissue disrupter 138B including the outer cutting edge 168 may have a transverse cross-section in the shape of a triangle, with the vertices of the triangle forming the upper inner edge 160, the lower inner edge 162, and the outer cutting edge 168 respectively.

As discussed above, it may be desirable in some implementations to avoid providing an outwardly extending cutting edge on the most distal curvature of the hoop 156. To this end, the elongate body 152 of the tissue disrupter 138B may be configured with a varying transverse cross-section along its length so as to provide the outer cutting edge 168 along some but not the entirety of the outer perimeter of the hoop 156. For instance, the elongate body 152 of the tissue disrupter 138B may include an intermediate portion 174 forming a distal curvature of the hoop 156 and side portions 176 disposed on each side of the intermediate portion 174, such as extending between the intermediate portion 174 and the terminal ends 154 of the tissue disrupter 138B. The transverse-cross sectional shape of the side portions 176 may form the outer cutting edge 168 (e.g., have the triangle cross-sectional shape), and the transverse-cross sectional shape of the intermediate portion 174 may form a relatively dull outward facing curvature (e.g., similar to the tissue disrupter 138A discussed above).

FIG. 18 illustrates an example tissue disrupter 138C similar to the tissue disrupter 138A, but with the hoop 156 forming a semi-circular shape. In this way, the tissue disrupter 138C may provide varying profiles along the outer perimeter defined by the outward facing surface 166 and along the upper and lower inner edges 160, 162, which may enable increased maneuverability and precision of the tissue disrupter 138C when dislodging and/or cutting tissue in the disc space.

FIG. 19 illustrates an example tissue disrupter 138D in which the hoop 156 forms a circular shape and defines an inner cutting edge 178 extending radially inwards along the inner perimeter of the hoop 156 to assist in further cutting tissue placed within the inner perimeter of the hoop 156. More specifically, the tissue disrupter 138D may include an outward facing surface 180 defining an outer perimeter of the hoop 156, and may include an upper inward facing surface 182 and a lower inward facing surface 184 each extending along the inner perimeter of the hoop 156. Each of the upper inward facing surface 182 and a lower inward facing surface 184 may also extend inwardly from a top end and bottom end of the outward facing surface 180 respectively so as to define the inner cutting edge 178 extending along the inner perimeter of the hoop 156 therebetween. The hoop 156 of the tissue disrupter 138D may further define an upper edge 186 extending along the outer perimeter of the hoop 156 between the outward facing surface 180 and the upper inward facing surface 182, and may define a lower edge 188 extending along the outer perimeter of the hoop 156 between the outward facing surface 180 and the lower inward facing surface 184. In some implementations, the upper and lower edges 186, 188 may be relatively blunt as compared to the inner cutting edge 178.

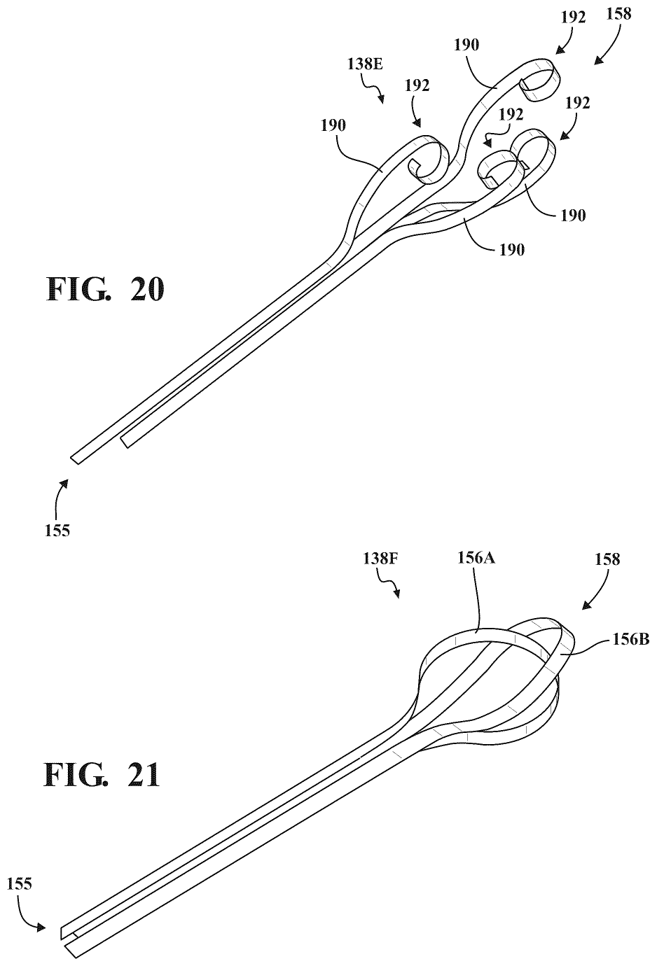

FIG. 20 illustrates another example tissue disrupter 138E forming a whisk-like open ribbon configuration. The tissue disrupter 138E may include a plurality of malleable protrusions 190 each extending distally from the proximal end portion 155 of the tissue disrupter 138E and terminating in a distal end portion 192 having a relatively blunt distal end when the tissue disrupter 138E is in the expanded configuration. For instance, as shown in the illustrated example, each of the protrusions 190 may include a strip of material, such as metal, having a distal end portion 192 configured to adjust from a straight configuration when the tissue disrupter 138E is in the collapsed state to curled configuration when the tissue disrupter 138E is in the expanded state, such as responsive to the outer cannula 122 being selectively and axially moved relative to the inner member 130 to place the tissue disrupter 138E in the expanded configuration as described above.

FIG. 21 illustrates a further example tissue disrupter 138F forming a whisk-like nested ribbon configuration. As shown in the illustrated example, tissue disrupter 138F may include a first hoop 156A and a second hoop 156B such that the first hoop 156A passes through and is transverse to the second hoop 156B in the expanded configuration.

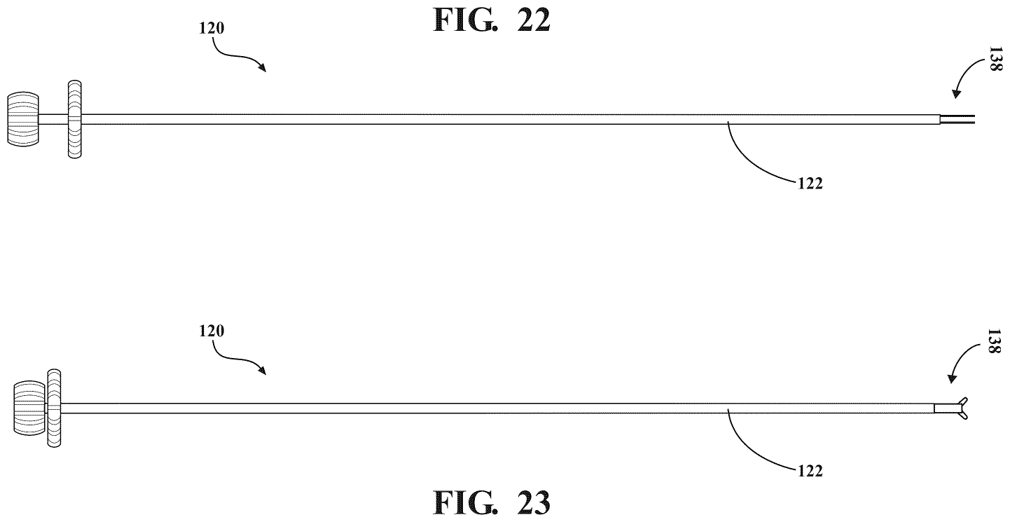

The various features of these ribbon-type tissue disrupters 138 may be configured to flex to multiple elongated shapes when in the collapsed configuration, such that multiple portions of the tissue disrupter 138 may be separately collapsed within the outer cannula 122. When placed in the expanded configuration such as illustrated in FIGS. 20 and 21, such configurations may provide for multiple edges or loops extending in varying directions to facilitate increased dislodging of the tissue. As an example, FIG. 22 illustrates a side view the tissue disrupter assembly 120 having a ribbon-type tissue disrupter 138 in the retracted state, and FIG. 23 illustrates a side view of the tissue disrupter assembly 120 of FIG. 22 in an extended state.

It should be noted that each of the examples shown in FIGS. 16-21 could be combined with other features and combinations. For example, at least a portion of the edges of the ribbon configurations of FIGS. 20 and 21 may be sharp, while others may be rounded.

It will be appreciated that the surgical access system and methods described herein have broad applications. The foregoing embodiments were chosen and described in order to illustrate principles of the methods and apparatuses as well as some practical applications. The preceding description enables others skilled in the art to utilize methods and apparatuses in various applications and with various modifications as are suited to the particular use contemplated. In accordance with the provisions of the patent statutes, the principles and modes of operation of this disclosure have been explained and illustrated in exemplary embodiments.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It will be understood that one or more steps within a method may be executed in a different order, may be executed serially or concurrently, and/or may include more or fewer steps than those illustrated and/or described above according to various aspects. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency. The terminology which has been used is intended to be in the nature of words of description rather than of limitation. Many modifications and variations are possible in light of the above teachings may be practiced otherwise than as specifically described.

What is claimed is:

1. An assembly for treating nucleus tissue of a spinal disc while reducing disruption to annulus tissue of the spinal disc, the assembly comprising
an outer cannula defined by a first open distal end, an opposite first proximal end, and a hollow body portion extending therebetween;
an inner member arranged at least partially within the outer cannula and having a second distal end and an opposite second proximal end; and
a tissue disrupter configured to dislodge tissue at a surgical site, the tissue disrupter fixed to and extending from the second distal end of the inner member,
wherein the outer cannula is selectively and axially movable relative to the inner member to adjust the tissue disrupter between a first state in which the tissue disrupter is in a collapsed configuration and arranged entirely within the outer cannula, and a second state in which the tissue disrupter is in an expanded configuration and positioned distal to the first open distal end, and
wherein the tissue disrupter forms a hoop in the expanded configuration, the hoop having at least one cutting edge to dislodge the nucleus tissue and a blunt distal curvature portion to reduce disruption of the annulus tissue while dislodging the nucleus tissue.

2. The assembly of claim 1, wherein the hoop forms a circular shape in the expanded configuration.

3. The assembly of claim 1, wherein the hoop forms a semi-circular shape in the expanded configuration.

4. The assembly of claim 1, wherein the hoop comprise opposed upper and lower inner edges each extending along an inner perimeter of the hoop.

5. The assembly of claim 4, wherein the hoop comprises an inward facing surface defining the inner perimeter of the hoop and an outward facing surface defining an outer perimeter of the hoop, wherein the upper and lower inner edges are defined at intersections of the inward and outward facing surfaces, and the outer facing surface is curved along a direction transverse to a length of the outer perimeter of the hoop to define the blunt distal curvature portion of the hoop.

6. The assembly of claim 1, wherein the tissue disrupter comprises an elongated body extending between first and second terminal ends positioned proximate each other to form the hoop at a distal end portion of the tissue disrupter, the elongated body including a first cross-sectional shape at an intermediate portion and a second cross-sectional shape at second portions adjacent the intermediate portion, the first cross-sectional shape defining the blunt distal curvature portion of the hoop and the second cross-sectional shape defining an outer cutting edge extending radially outward along an outer perimeter of the hoop.

7. The assembly of claim 1, wherein the hoop defines an inner cutting edge extending radially inward along an inner perimeter of the hoop.

8. The assembly of claim 1, wherein the tissue disrupter forms a first hoop and a second hoop in the expanded configuration, the first hoop passing through and being transverse to the second hoop.

9. The assembly of claim 1, wherein the tissue disrupter is formed of malleable material and configured to bend around bone.

10. The assembly of claim 1, wherein the tissue disrupter is bendable and configured to collapse into the collapsed configuration as a direct result of the outer cannula being moved over the tissue disrupter.

11. The assembly of claim 10, wherein the tissue disrupter is semi-rigid in the expanded configuration.

12. The assembly of claim 1, further comprising a first hub arranged at the first proximal end of the outer cannula and a second hub arranged at the second proximal end of the inner member, wherein the first hub is spaced from the second hub in the first state, wherein the inner member is of greater length than the outer cannula, and wherein the second hub is configured to selectively move along the inner member to abut the first hub to place the tissue disrupter in the second state.

13. An assembly comprising:
an outer cannula defined by a first open distal end, an opposite first proximal end, and a hollow body portion extending therebetween;
an inner member arranged at least partially within the outer cannula and having a second distal end and an opposite second proximal end; and
a tissue disrupter configured to dislodge tissue at a surgical site, the tissue disrupter fixed to and extending from the second distal end of the inner member,
wherein the outer cannula is selectively and axially movable relative to the inner member to adjust the tissue disrupter between a first state in which the tissue disrupter is in a collapsed configuration and arranged entirely within the outer cannula, and a second state in which the tissue disrupter is in an expanded configuration and positioned distal to the first open distal end,
wherein the tissue disrupter includes a plurality of malleable protrusions each extending distally from the second distal end of the inner member and terminating in a blunt distal end when the tissue disrupter is in the expanded configuration.

14. The assembly of claim 13, wherein each of the protrusions comprises a material strip having a distal end portion configured to curl into a loop to form the blunt distal end responsive to the outer cannula being selectively and axially moved relative to the inner member to place the tissue disrupter in the second state.

15. The assembly of claim 14, wherein the curled loops are disposed in varying planes.

16. A method for treating nucleus tissue of a spinal disc while reducing disruption to annulus tissue of the spinal disc, the method comprising:
providing an access portal to the nucleus tissue of the spinal disc;
obtaining a tissue disrupter assembly comprising an outer cannula defined by a first open distal end, an opposite first proximal end, and a hollow body portion extending therebetween; an inner member arranged at least partially within the outer cannula and having a second distal end and an opposite second proximal end; and a tissue disrupter fixed to and extending from the second distal end of the inner member, the tissue disrupter being in a first state in which the tissue disrupter is in a collapsed configuration and arranged entirely within the outer cannula;
inserting the obtained tissue disrupter assembly in the first state through the access portal such that the first open distal end of the outer cannula passes through the annulus tissue and is disposed in the nucleus tissue;
adjusting the tissue disrupter to a second state in which the tissue disrupter is in an expanded configuration and positioned distal to the first open distal end by moving the outer cannula relative to the inner member in a proximal direction, wherein the tissue disrupter forms a hoop in the expanded configuration that has at least one cutting edge to dislodge the nucleus tissue and a blunt distal curvature portion to reduce disruption of the annulus tissue while dislodging the nucleus tissue;
dislodging the nucleus tissue from vertebrae adjacent the spinal disc by moving the tissue disruption assembly in the second state within the spinal disc;
after dislodging the nucleus tissue, adjusting the tissue disrupter back to the first state by moving the outer cannula relative to the inner member in a distal direction;
withdrawing the tissue disrupter back in the first state from the access portal; and
removing the dislodged nucleus tissue from the spinal disc using a tissue removal device inserted through the access portal.

17. The method of claim 16, wherein providing the access portal to the nucleus tissue of the spinal disc comprises inserting an endoscope with a working channel through the annulus tissue of the spinal disc, the working channel defining the access portal.

18. The method of claim 16, comprising inserting the tissue disrupter assembly into the spinal disc and withdrawing the tissue disrupter from the spinal disc only once during the treatment of the nucleus tissue.

19. The method of claim 16, wherein the hoop comprises an upper inner edge and a lower inner edge each extending along an inner perimeter of the hoop, and dislodging the nucleus tissue from the vertebrae adjacent the spinal disc comprises scraping the nucleus tissue from the vertebrae using the upper and lower inner edges.

20. The method of claim 16, wherein the tissue disrupter forms a first hoop and a second hoop in the expanded configuration, the first hoop passing through and being transverse to the second hoop, and the method comprising dislodging the nucleus tissue from the vertebrae adjacent the spinal disc by rotating the tissue disrupter assembly within the nucleus tissue.
